(19) United States
(12) Patent Application Publication
(10) Pub. No.: US 2014/0249636 A1
(43) Pub. Date: Sep. 4, 2014

(54) FORCEPS FOR GRIPPING AN INTERVERTEBRAL IMPLANT, A KIT, AND A MANIPULATOR ASSEMBLY FOR MANIPULATING SUCH FORCEPS

(71) Applicant: LES LABORATOIRES OSTEAL MEDICAL, Roissy Aeroport Cdg (FR)

(72) Inventors: Daniel Bouchot, Noisiel (FR); Yoachim Horyn, Saint Mande (FR); Thierry Tisserand, Aulnay Sous Bois (FR)

(73) Assignee: LES LABORATOIRES OSTEAL MEDICAL, Roissy Aeroport Cdg (FR)

(21) Appl. No.: 14/351,689

(22) PCT Filed: Oct. 16, 2012
(86) PCT No.: PCT/FR2012/052356

§ 371 Date: Apr. 14, 2014

(30) Foreign Application Priority Data
Oct. 17, 2011 (FR) 1159371

(51) Int. Cl. A61F 2/46

(52) U.S. Cl. CPC A61F 2/4611; A61F 2002/4615; A61F 2002/4619
USPC 623/17.16

(57) ABSTRACT

A forceps (1) for gripping an intervertebral implant (2), said forceps comprising two rigid arms (11) that are hinged relative to one another at a connection member (10), the two arms (11) defining between them an internal space (120), each arm (11) forming a jaw (17), the two jaws (17) being for coming into engagement with an intervertebral implant (2), the jaws (17) being movable between an open position and a closed position in clamping engagement with the implant (2), at least one of the two arms (11) including a cam surface (16) for urging the jaws (17) in the open position;
said forceps being characterized in that the cam surface (16) extends outside the internal space (120) defined between the arms (11).

The present invention further provides an implantation kit and an implantation assembly comprising said forceps and an intervertebral implant.

The present invention relates to a forceps for gripping an intervertebral implant (or intervertebral disk prosthesis), which forceps makes it possible to take hold of, in stable and unitary manner, an intervertebral implant for inserting between two vertebrae of a vertebral column in order to replace a deteriorated natural intervertebral disk. The present invention also relates to an implantation kit comprising forceps of the invention and an intervertebral implant engaged in the forceps. The invention further relates to an implantation assembly comprising a forceps of the invention, an intervertebral implant engaged in the forceps, and a manipulator tool that is adapted to take hold of the forceps and to move the jaws of the forceps between closed and open positions. The field of application of the present invention is naturally the field of surgical accessories used to put an intervertebral implant into place between two vertebrae of a vertebral column.

Document WO 2008/021645 is known that describes an instrument for inserting an implant. With reference to FIG. 20 of that document, the instrument can be seen in an exploded view. The implant is designed to be held by two jaws. The jaws are mounted on two arms, each including a sloping cam surface. The arms are for engaging inside a rod that slides inside a sleeve. Thus, by retracting the rod into the sleeve, the sloping cam surfaces come into engagement with the inner edge of the open end of the sleeve, and thus urge the jaws in engagement with the implant. Consequently, the cam surfaces do not have the function of urging the jaws in the open position, but, on the contrary, in the closed position. Specifically, it is not possible for cam surfaces that are oriented outwards to be driven in such a manner as to urge the jaws into the open position.

In the prior art, document FR 2 893 839 is already known that describes an insertion instrument for inserting an intervertebral implant between two vertebrae. The insertion instrument includes a rod that is provided with two tabs that form a forceps and that slide in a tube. Sliding towards the grip end causes the forceps to close by the outsides of the tabs on the rod making contact with a frustoconical portion of the tube, and sliding towards the manipulation end causes the forceps to open by a spacer pin of the tube making contact with the insides of the tabs of the rod. The two tabs are connected together via a hinge pin that is secured to the rod. In that prior-art insertion instrument, the forceps formed by the two tabs and its hinge pin forms an integral part of the insertion instrument, which is also constituted by the rod, the tube, and its spacer pin. As a result, it is not possible to disassociate the tabs from the rod and/or from the tube. In addition, it should be observed that the forceps is closed and locked by retracting the tabs inside the tube, while the forceps is opened by extending the tabs out from the tube against its spacer pin. The forceps is thus closed and opened by moving the forceps in opposite directions.

An object of the present invention is to propose an insertion instrument that makes it possible to take hold of a prosthesis or intervertebral implant substantially in the same way as in document FR 2 893 839. However, the insertion instrument of the present invention presents a structure that is different to the structure of the above-mentioned document, that enables the forceps to be removed or replaced easily, and that further presents a configuration that is particularly simple, such that the forceps may be disposable, i.e. for single use only. Another object of the present invention is to simplify the mode of operating the insertion instrument, and in particular the mode of moving the rod relative to the tube. Still another object of the present invention is to eliminate the spacer pin for moving the forceps apart, which spacer pin is at the origin of the complicated structure of the instrument in document FR 2 893 839. In other words, the present invention seeks to separate the forceps from its manipulator tool, so that the forceps can be packaged in the form of a kit with the intervertebral implant already engaged in the forceps. Thus, the forceps is associated with the intervertebral implant and not with the tool for manipulating the forceps, and, as a result, the forceps may be disposable.

To achieve these objects, the present invention proposes a forceps for gripping an intervertebral implant, said forceps comprising two rigid arms that are hinged relative to one another at a connection member, the two arms defining between them an internal space, each arm forming a jaw, the two jaws being for coming into engagement with an intervertebral implant, the jaws being movable between an open position and a closed position in clamping engagement with the implant, at least one of the two arms including a cam surface for urging the jaws in the open position, said forceps being characterized in that the cam surface extends outside the internal space defined between the arms.

In the above-mentioned prior-art document FR 2 893 839, the cam surfaces are formed by the inner faces of the arms, while the locking profiles are formed by the outer faces of the arms. In the present invention, the cam surfaces are situated outside the arms, so that it is possible to eliminate the prior art spacer pin for moving the arms apart. By positioning the cam surface(s) outside the locking profile, it is possible to connect the connection member of the forceps to the rod of the manipulator tool in very simple manner, given that it is not necessary to engage the arms of the forceps around a spacer pin. In one of its main aspects, the present invention may thus be seen as moving the cam surface(s) to outside the arms, compared to the above-mentioned prior art document FR 2 893 839.

In an advantageous aspect of the present invention, said at least one arm forms an external housing that is situated outside the internal space, the external housing forming the cam surface. The housing is thus situated outside the arm, and not between the arms.

According to an optional characteristic of the invention, one or each arm further includes a locking profile for locking in the closed position. Advantageously, the locking profile and the cam surface of an arm point towards each other. This does not mean that the cam surface is arranged directly facing or in register with the locking profile: this merely means that the cam surface faces generally towards the locking profile in substantially converging manner. In the above-mentioned prior-art document, the cam surface and the locking profile also point in opposite directions, but away from each other.

According to another advantageous characteristic of the present invention, at least one of the two arms includes a branch and a tab that extends approximately parallel to the branch, the branch and the tab forming between them an external housing that is open towards the connection member, the tab forming the cam surface, and the branch advantageously forming a locking profile for locking in the closed position. Thus, when the forceps includes two cam surfaces and two locking profiles, each pair formed by a locking profile and a cam surface is situated in a respective housing, such that each pair may be actuated by a single contact member, as described below.

In a practical embodiment, the connection member includes removable connection means that are adapted to connect the forceps in removable manner to a manipulator tool that is adapted to take hold of the forceps and to move its jaws between the closed and open positions. Thus, the forceps does not form an integral part of the manipulator tool, but constitutes an independent accessory that may be combined with the implant so as to constitute an implantation kit.

In another aspect of the invention, the forceps is made as a single piece, the arms being connected to the connection member via a flexible connection. Thus, by way of example, the forceps of the invention may be made by injection molding an appropriate plastics material. It may also be made by machining or by extruding, e.g. metal.

In summary, in a preferred embodiment, the forceps of the invention includes two hinged arms, each forming a housing facing outwards and defining a cam surface and advantageously a locking profile. The forceps is made as a single piece that is separate from the manipulator tool, such that the forceps may be associated with an intervertebral implant already engaged in the forceps, so as to constitute an implantation kit that may be packaged in sterile manner. Preferably, when in the rest state, the forceps is in the closed position, so as to hold the intervertebral implant. However, in the absence of external stress, the forceps could equally well be in the open position.

The present invention also defines an implantation assembly comprising a forceps as defined above, an intervertebral implant engaged in the forceps, and a manipulator tool that is adapted to take hold of the forceps and to move its jaws between the closed and open positions, the manipulator tool comprising:
a tube that is provided with a handle;
a rod that is slidably mounted in the tube to slide along a slide axis X, the rod being connected to the connection member of the forceps; and
a mechanism for moving the rod in the tube along the slide axis X;

the implantation assembly being characterized in that the tube of the tool includes at least one contact member that comes into engagement with the cam surface of the forceps while the rod is sliding in the tube. When the forceps includes two cam surfaces, the tube forms two corresponding contact members that are situated on either side of the rod, outside the internal space formed by the forceps.

According to an advantageous characteristic of the implantation assembly, the rod includes a connection profile that is connected in removable manner to the connection member of the forceps, the rod slides in the tube between an extended position in which the connection member of the forceps is engageable on the connection profile of the rod that thus projects out from the tube, and a retracted position in which the contact member is in engagement with the cam surface of the forceps. The forceps may thus be put into place and removed from the tool easily, without being hindered by the contact members.

According to another characteristic of the invention, the rod is movable into an intermediate locking position that is situated between the extended and retracted positions, and in which the contact member is thus in engagement with a locking profile that is formed by an arm of the forceps. Thus, it is a single contact member that actuates a pair constituted by a locking profile and a cam surface. Since the forceps of the invention preferably includes two locking profile and cam surface pairs, there are two contact members. It should also be observed that by retracting the rod inside the tube from the extended position, the rod passes via the intermediate locking position before arriving at the completely-retracted final position. In other words, the step of mounting the forceps on the tool, the step of locking the forceps in the closed position, and the step of opening the forceps are performed by moving the rod inside the tube in a single direction. In practice, the manipulator tool is initially brought into the extended position in such a manner that the implantation kit (forceps plus intervertebral implant) can be connected to the manipulator tool. Then, the user actuates the mechanism for moving the rod inside the tube in such a manner as to retract the rod into the tube until it reaches the intermediate locking position. In this position, the forceps is in blocked engagement on the intervertebral implant that may then be inserted or implanted between two vertebrae. Once the implantation operation is terminated, the operator retracts the rod even further into the tube, in such a manner as to reach the retracted position in which the forceps is urged into the open position. It may then be disengaged from the intervertebral implant and removed together with the manipulator tool.

In another practical aspect of the invention, the tool includes indicator means for indicating to the user that the rod is in the intermediate locking position. In this way, the user of the manipulator tool can be certain that the forceps is locked on the intervertebral implant.

According to another characteristic of the invention, said at least one contact member of the tube is in the form of a bar having an outer face that comes into engagement with the cam surface, and having an inner face that advantageously comes into engagement with a locking profile of the arm of the forceps. It is thus the two faces of a single member that come successively into engagement with a locking profile and with a cam surface.

In another practical aspect of the invention, the connection member of the forceps is engaged laterally or axially on the connection profile of the manipulator tool in the extended position of the rod, the connection between the connection member and the connection profile being locked by inserting the connection profile into the tube, the connection profile advantageously being elastically deformable so as to guarantee the connection member is held on the connection profile, even before it is inserted into the tube. The forceps is put into place on the tool more easily as a result of there being no element of the tool that is positioned between the arms of the forceps. Specifically, there is no spacer pin as in document FR 2 893 839.

One of the principles of the present invention resides in having the cam surface(s) of the forceps outside the arms, advantageously in the proximity of a possible locking profile, such that they may both be actuated as a pair by a single contact member. The rod may thus be moved inside the tube in unidirectional manner from the extended position (putting the forceps into place) towards the completely retracted position (moving the jaws apart), optionally passing via an intermediate locking position.

The invention is described more fully below with reference to the accompanying drawings, which show an embodiment of the invention by way of non-limiting example.

In the figures:

FIG. 1 is a plan view of a forceps of the invention in engagement with an intervertebral implant;

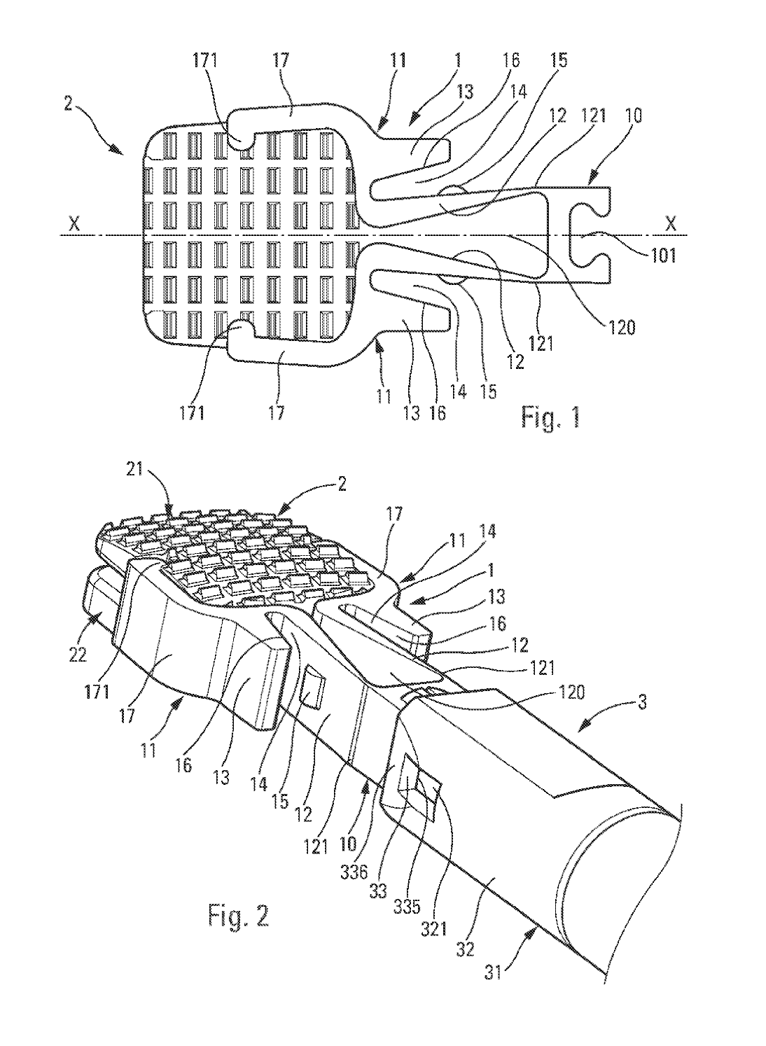

FIG. 2 is a perspective view of the FIG. 1 forceps (and of the implant) connected to a manipulator tool of the invention;

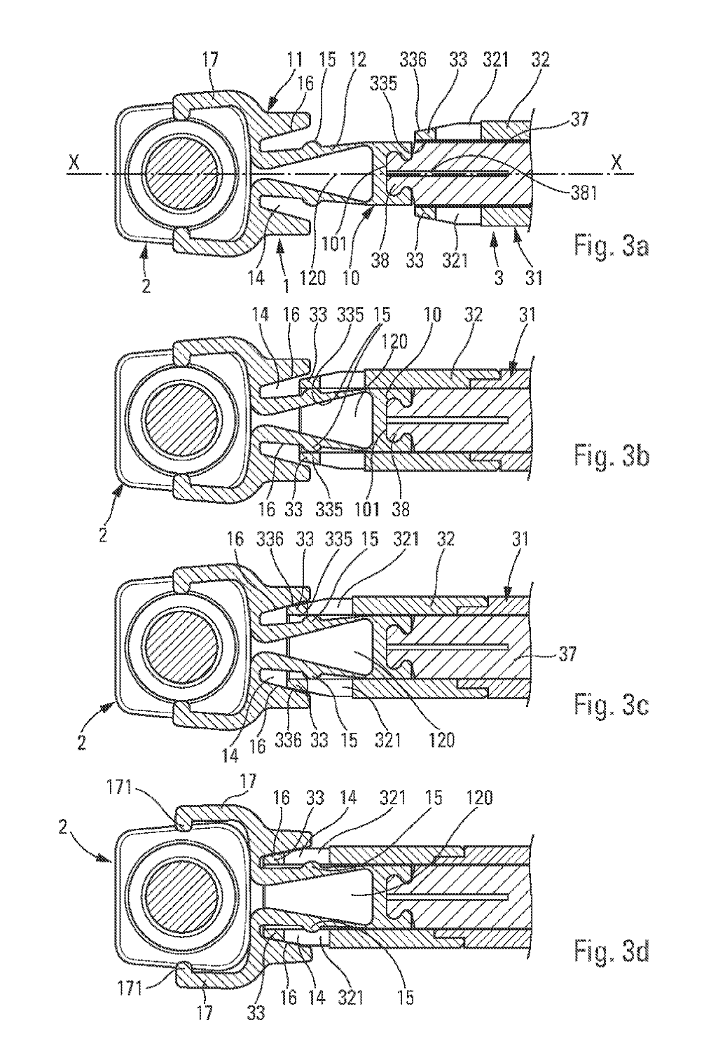

FIGS. 3a to 3d are horizontal section views through an implant, a forceps, and a tool of the invention in four functional configurations showing how the forceps and the manipulator tool are operated;

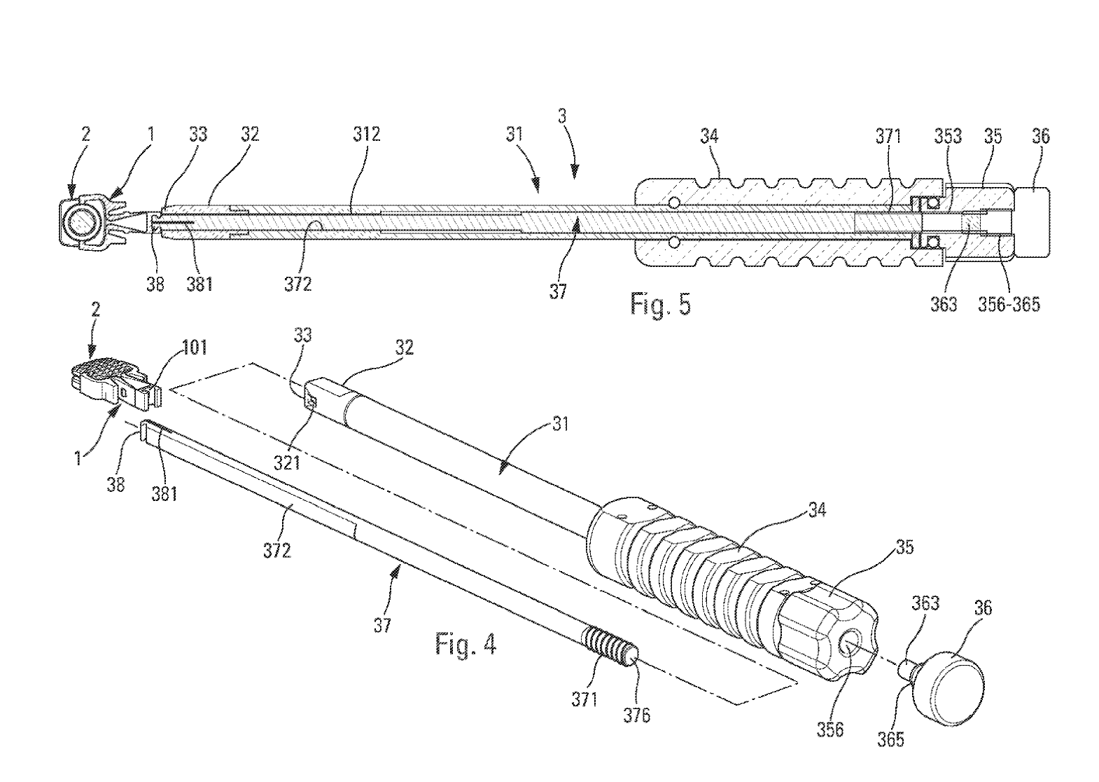

FIG. 4 is an exploded perspective view of a manipulator tool of the invention ready to be connected to an implantation kit formed by a forceps of the invention together with an intervertebral implant; and

FIG. 5 is a longitudinal section view through the FIG. 4 manipulator tool connected to a forceps of the invention in engagement with an intervertebral implant.

Reference is made firstly to FIGS. 1 and 2 in order to describe in detail the structure of a forceps of the invention. The forceps, designated overall by the numerical reference 1, is preferably made as a single piece by molding or injection molding plastics material, or by machining or extruding metal, e.g. titanium for machining, or aluminum for extruding. The forceps 1 includes two arms 11 that are preferably identical as mirror images about the axis X shown in FIG. 1. The two arms 11 are connected to a connection member 10 that forms connection means 101 in the form of a connection housing. In the embodiment shown in the figures, the connection housing is a female housing, but a male housing would also be suitable. The connection member 10 is connected via two opposite edges to the two arms 11 via elastically-deformable connections 121 that are made in the form of respective reductions in wall thickness, imparting flexibility or elastic resilience having shape memory. Thus, the two arms 11 always return to their initial rest position after being subjected to stress. Without going beyond the ambit of the invention, provision can also be made for the flexible connections 121 not to be resilient, such that after deformation, the arms 11 do not return to their initial rest position. Preferably, the arms 11 are rigid, so that the deformation of the forceps is localized in the flexible connections 121 only. It is thus possible to make the forceps with a stationary arm and a movable arm: in this configuration, only the movable arm is connected to the connection member via a flexible connection, the stationary arm being connected to the connection member in stationary and solid manner.

Each arm 11 includes a branch 12 that is connected at one of its ends to the connection member 10 via the flexible connection 121. The branches 12 extend substantially parallel to the axis X, while nevertheless converging towards each other on going away from the connection member 10. They could also be completely parallel. Between them, they define an internal space 120. At their other ends, the branches 12 are extended to form two jaws 17 that are provided with gripper teeth (or studs) 171 for coming into engagement with an intervertebral implant 2. The implant 2 may comprise a top plate 21 and a bottom plate 22 that are movable relative to each other on a core, thereby reproducing the movements that are possible for two vertebrae that are separated by a natural intervertebral disk. The jaws 17 of the forceps are designed for receiving the implant 2 in stable and stationary manner. More particularly, the gripper teeth 171 may come into engagement with the top plate 21 and with the bottom plate 22 and hold them stationary relative to each other. By way of example, the teeth 171 may extend over the top plate 21 and under the bottom plate 22, and between the two plates so as to hold them firmly.

Each arm 11 also includes a tab (or lug) 13 that extends from the jaw 17 substantially parallel to the branch 12, in such a manner as to define a free end that points generally towards the connection member 10. The tab 13 may present a base that is reinforced relative to its free end, in such a manner as to present a trapezoidal or wedge shape. The tab 13 defines an outer face that faces away from the branch 12, and an inner face that faces towards the branch 12. An external housing 14 is thus formed between the branch 12 and the tab 13, which housing is open towards the connection member 10. The housing 14 is situated outside the internal space 120 defined between the two branches 12. Preferably, both branches 11 are formed with such tabs forming external housings 14. However, a forceps of the invention could be formed with only one tab 13 and only one external housing 14, the other tab not being provided. In this configuration, the forceps is opened by moving only one jaw. The other jaw may be stationary.

In the invention, the inner face of the tab 13 forms a cam surface 16 that slopes relative to the axis X of the forceps 1. More precisely, the cam surface 16 goes towards the axis X on penetrating into the external housing 14. Thus, by engaging a contact member into the external housing 14 along an axis that is parallel to the axis X, the contact member comes into sliding contact against the cam surface 16, which generates movement of the arm 11 by pivoting about the flexible connection 121. As a result, the gripper teeth 171 disengage the intervertebral implant 2. By providing one contact member per external housing 14, it is possible to move or to urge the arms 11 of the forceps into an open position, making it possible to remove the forceps easily from the implant 2. In the rest state, the forceps 1 is preferably in the closed position, as shown in FIG. 1. However, it is also possible to envisage a forceps that is open in the rest state. In this configuration, engaging the contact member with the cam surfaces 16 merely guarantees that the forceps is opened in the event that the jaws 17 are impeded by anything in the environment. However, the forceps is preferably in the closed position in the rest state, thereby making it possible to take hold of the intervertebral implant 2 without having to exert external stress on the forceps.

According to another advantageous characteristic of the invention, each of the outer faces of the arms 12 forms a locking profile 15 in the form of a protuberance or projection that extends outwards, substantially level with the free end of the corresponding tab 13. The locking profiles 15 may thus be considered as being situated in the external housings 14, or directly at their inlets. The function of the locking profiles 15 is to block or to lock the forceps 1 in the closed position shown in FIGS. 1 and 2 by means of the contact member that then comes into engagement with the cam surfaces 16. The locking profiles 15 may also be used to move the jaws into the closed position. The locking profiles 15 constitute an optional characteristic that may be omitted, particularly when the forceps is in the closed position in the rest state. However, the locking profiles are an advantageous characteristic that guarantees that the forceps is locked properly in the closed position. In this configuration, it should be observed that for the external housing under consideration, the locking profile 15 points substantially towards the facing cam surface 16. It should also be observed that, like the cam surfaces 16, the locking profiles 15 are situated outside the internal space 120 defined between the two branches 11. In another possible definition, it can be said that each external housing 14 forms a cam surface 16 and optionally a locking profile 15. It can also be said that the tabs or lugs 13 are arranged on either side of the branches 12 outside the internal housing 120. This characteristic is particularly advantageous given that it is not necessary to insert any member between the two branches 12 in order to move the arms 11 apart. It is thus possible to move the arms 11 from the closed position towards the open position from outside the branches 12, by inserting respective contact members into the external housings 14. It is possible to envisage making a forceps with a single locking profile. One arm may be movable while the other is stationary. The locking profile and the cam surface would then be provided on the movable arm only.

Reference is made below to FIGS. 3a to 3d which show how the forceps 1 may be urged into the open state and how the forceps may optionally be locked in the closed state by using a manipulator tool 3, of which only one end is shown. The manipulator tool 3 is described in detail below with reference to FIGS. 4 and 5. In brief, the manipulator tool 3 includes a hollow tube 31 that is provided with an endpiece 32 that forms two contact bars 33 that are defined by two windows 321. The manipulator tool 3 also includes a rod 37 that is slidably engaged inside the tube 31 and the endpiece 32, along the axis X that coincides with the axis of mirror symmetry of the forceps 1. The end of the rod 37 is formed with a connection profile 38 that, in this embodiment, is a male profile for engaging inside the female connection housing 101 of the connection member 10 of the forceps 1. Instead of this male connection head, alternatively, a female connection housing could be provided, and in this configuration the connection member 10 could be formed with a male connection head. In the configuration shown in the figures, the male connection profile 38 of the rod 37 is advantageously formed with a slot 381, thereby imparting a certain amount of springiness thereto. In this way, it is possible to engage the connection profile 38 in the female housing 101, in such a manner as to ensure the profile 38 is held resiliently inside the housing 101. In this way, the forceps may be held temporarily on the rod 37. This step of engaging the forceps 1 on the rod 37 is shown in FIG. 3a. It can be seen that the male connection profile 38 of the rod 37 thus projects out beyond the end of the endpiece 32 formed with the two bars 33. The forceps is put into place on the rod 37 by lateral engagement along the axis X, which is also the sliding axis of the rod 37 inside the endpiece 32. It is also possible to envisage engaging the forceps axially on the rod with snap fastening. Once the forceps 1 is in place in this way on the rod 37, the rod may be slid towards the inside of the tube 31 and the endpiece 32, such that the connection profile 38 and the connection member 10 penetrate inside the endpiece 32, as can be seen in FIG. 3b. It can easily be understood that the connection between the forceps 1 and the rod 37 is thus locked by the endpiece 32 that surrounds them. It should also be observed that the two bars 33 are thus situated level with the two locking profiles 15, at the inlets to the external housings 14, thereby preventing the arms 11 of the forceps from moving apart. More precisely, each bar 33 includes an inner face 335 that may come into contact with the projection forming the locking profile 15. It should also be observed that the two bars 33 are also not in contact with the cam surfaces 16. In this intermediate position, not only is the forceps stationary and securely attached to the rod 11, but also both arms 11 of the forceps are locked in the closed position. It is thus possible to manipulate the forceps 1 and the intervertebral implant 2 without risk of losing the implant or of breaking the connection between the forceps and the manipulator tool 3. The manipulator tool 3, the forceps 1, and the intervertebral implant 2 thus constitute a unitary implantation assembly that may be manipulated by a surgeon so as to put the implant 2 into place between two vertebrae of a vertebral column. Once the implant is in place between the two vertebrae, it is necessary to withdraw the forceps 1 from the implant 2, and this is done by moving the two jaws 17 of the arms 11 apart. To do this, the rod 37 is moved further inside the tube 31, so that the bars 33 come into contact with the cam surfaces 16 of the forceps. This is shown in FIG. 3c. More precisely, each bar 33 defines an outer face 336 that comes into sliding contact with a respective cam surface 16. Given that the rod 37 moves axially along the axis X and that the cam surfaces 16 slope relative to that axis X, the outer faces 336 of the bars 33 urge the arms 11 of the forceps to move apart as the bars move towards the bottoms of the external housings 14. This final retracted position is shown in FIG. 3d. The gripper teeth 171 are disengaged from the intervertebral implant 2, and the forceps 1 may thus be removed from the implant that remains in place between the two vertebrae. The implantation operation proper is thus terminated.

The two bars 33 constitute contact members having the function of coming into engagement with the or each cam surfaces 16, and optionally with the locking profiles 15. Thus, a single contact member performs two functions, namely a function of moving at least one arm in order to move the arms apart, and a function of locking the arms in the closed position. It should be observed that the contact member is situated outside the internal space 120. The various manipulation steps in FIGS. 3a to 3d are performed by moving the rod 37 in a single direction, namely towards the inside of the tube 31. Thus, it is not necessary to reverse the direction in which the rod 37 is moved while implanting the intervertebral implant 2.

It should also be observed that the bars 33 are never simultaneously in contact with the locking profiles 15 and with the cam surfaces 16. This can be seen in FIG. 3c. The bar 33 passes successively from contact with the locking profiles 15 to contact with the cam surfaces 16, thereby avoiding any blockage or hard point in manipulation.

Reference is made below to FIGS. 4 and 5 in order to describe in greater detail the manipulator tool 3. As mentioned above, the manipulator tool 3 includes a rod 37 that is slidaby movable inside a tube 31 that is provided, at its end, with an endpiece 32 that forms the two bars that act as contact members. In order to move the rod 37 inside the tube 31, a mechanism is provided that makes it possible to transform a turning movement into a movement in translation, like the movements used in lipsticks. To do this, at its end remote from the endpiece 32, the tube 31 is provided with a knurled wheel 35 that is mounted to turn freely. Internally, the knurled wheel 35 includes first tapping 353 that engages with a threaded end 371 of the rod 37 remote from the connection profile 38. In addition, the rod 37 is formed with one or two flats 372 that are for sliding against two plane walls 312 formed inside the tube 31. Thus, the rod 37 is prevented from turning inside the tube 31. Thus, by actuating the knurled wheel 35, the threaded end 371 of the rod 37 is constrained to move in the tapping 353 of the knurled wheel 35. Thus, turning movement of the knurled wheel 35 is transformed into movement of the rod 37 in axial translation inside the tube 31. In order to hold the tube 31 in the hand, a handle 34 is advantageously provided.

According to an advantageous characteristic, the knurled wheel 35 is provided with an abutment head 36 that forms an abutment pin 363 and a threaded section 365. In addition, the knurled wheel 35 includes second tapping 356 in which the thread 365 of the abutment head 36 is engaged. In FIG. 5, the abutment head is fully tightened in the knurled wheel 35, such that the pin 363 is fully engaged inside the knurled wheel. Thus, it can readily be understood that by turning the knurled wheel 35, the rod 37 moves inside the tube 31 and the knurled wheel 35 until it comes into abutment against the pin 363 of the abutment head 36. The abutment position corresponds to the intermediate locking position shown in FIG. 3c. In other words, when the abutment head 36 is fully engaged on the knurled wheel 35, the operator or the surgeon knows that by turning the knurled wheel 35 to the maximum, the intermediate locking position will be reached, in which position the implantation assembly may be used to put the intervertebral implant 2 into place between two vertebrae of a vertebral column. Once the implant is in place, the operator or the surgeon unscrews the abutment head 36 and then turns the knurled wheel 35 so as to reach the retracted position of FIG. 3d in which the forceps 1 is in the open position. Thus, the abutment head 36 serves as physical indicator means enabling the user to know that the implantation assembly is ready for putting the implant 2 into place between two vertebrae. Instead of or in addition to the abutment head 36, it is also possible to provide visual indicators, e.g. marking at the endpiece 32.

In this embodiment, the tube 31 is made in two pieces, but alternatively it could be made as a single piece, or on the contrary out of three pieces or even more. The rod 37 is prevented from turning in the tube 31 over the length of the tube 31, but alternatively this blocking function could be provided at the endpiece 32 only. The endpiece 32 includes two contact bars 33, but alternatively only a single bar need be provided when only one arm 11 is hinged. In addition, without going beyond the ambit of the invention, the cam surface and the contact member may be inverted, such that the cam surface would be formed by the tube, and the contact member that comes into sliding contact on the cam surface would be formed by the forceps. This would merely be an inversion of characteristics, without modifying the technical function.

Positioning the cam surfaces 16 outside the branches 12 makes it possible to move the arms 11 apart without having to access the internal space 120 defined between the two arms 12. As a result, putting the forceps 1 into place on the manipulator tool 3 and removing it therefrom is extremely easy, given that the contact members 33 are completely inoperative and set back while the forceps is being put into place on the end of the rod 37. The forceps 1 thus constitutes an element that can be separated from the manipulator tool 3, which means that it can be associated with the intervertebral implant 2 in the form of an implantation kit, e.g. packaged in sterile manner in an appropriate package. Once the kit is put into place on the manipulator tool in accordance with the above-described mode of operating, the assembly constitutes an implantation assembly that can be manipulated in unitary manner until the intervertebral implant has been put into place between two vertebrae. The forceps may then be removed from the manipulator tool 3 and discarded as a single-usage accessory.

1. A forceps (1) for gripping an intervertebral implant (2), said forceps comprising two rigid arms (11) that are hinged relative to one another at a connection member (10), the two arms (11) defining between them an internal space (120), each arm (11) forming a jaw (17), the two jaws (17) being for coming into engagement with an intervertebral implant (2), the jaws (17) being movable between an open position and a closed position in clamping engagement with the implant (2), at least one of the two arms (11) including a cam surface (16) for urging the jaws (17) in the open position;
said forceps being characterized in that the cam surface (16) extends outside the internal space (120) defined between the arms (11).

2. A forceps (1) according to claim 1, wherein said at least one arm (11) forms an external housing (14) that is situated outside the internal space (120), the external housing (14) forming the cam surface (16).

3. A forceps (1) according to claim 1, wherein at least one of the two arms (11) includes a locking profile (15) for locking in the closed position.

4. A forceps (1) according to claim 3, wherein the locking profile (15) and the cam surface (16) of an arm (11) point towards each other.

5. A forceps (1) according to claim 1, wherein at least one of the two arms (11) includes a branch (12) and a tab (13) that extends approximately parallel to the branch (12), the branch (12) and the tab (13) forming between them an external housing (14) that is open towards the connection member (10), the tab (13) forming the cam surface (16), and the branch (12) advantageously forming a locking profile (15) for locking in the closed position.

6. A forceps (1) according to claim 1, wherein the connection member (10) includes removable connection means (101) that are adapted to connect the forceps (1) in removable manner to a manipulator tool (3) that is adapted to take hold of the forceps (1) and to move its jaws (17) between the closed and open positions.

7. A forceps (1) according to claim 1, made as a single piece, the arms (11) being connected to the connection member (10) via a flexible connection (121).

8. An implantation kit comprising a forceps (1) according to claim 1, and an intervertebral implant (2) engaged in the forceps (1).

9. An implantation assembly comprising a forceps (1) according to claim 1, an intervertebral implant (2) engaged in the forceps, and a manipulator tool (3) that is adapted to take hold of the forceps (1) and to move its jaws (17) between the closed and open positions, the manipulator tool (3) comprising:
a tube (31) that is provided with a handle (34);
a rod (37) that is slidably mounted in the tube (31) to slide along a slide axis X, the rod (37) being connected to the connection member (10) of the forceps; and
a mechanism (35) for moving the rod (37) in the tube (31) along the slide axis X;
the implantation assembly being characterized in that the tube (31) of the tool (3) includes at least one contact member (33) that comes into engagement with the cam surface (16) of the forceps while the rod (37) is sliding in the tube (31).

10. An implantation assembly according to claim 9, wherein the rod (37) includes a connection profile (38) that is connected in removable manner to the connection member (10) of the forceps, the rod (37) slides in the tube (31) between an extended position in which the connection member (10) of the forceps is engageable on the connection profile (38) of the rod (37) that thus projects out from the tube (31), and a retracted position in which the contact member (33) is in engagement with the cam surface (16) of the forceps.

11. An implantation assembly according to claim 10, wherein the rod (37) is movable into an intermediate locking position that is situated between the extended and retracted positions, and in which the contact member (33) is thus in engagement with a locking profile (15) that is formed by an arm (11) of the forceps.

12. An implantation assembly according to claim 11, wherein the tool (3) includes indicator means (36) for indicating to the user that the rod (37) is in the intermediate locking position.

13. An implantation assembly according to claim 9, wherein said at least one contact member of the tube (31) is in the form of a bar (33) having an outer face (336) that comes into engagement with the cam surface (16), and having an inner face (335) that advantageously comes into engagement with a locking profile (15) of the arm (11) of the forceps.

14. An implantation assembly according to claim 10, wherein the connection member (10) of the forceps is engaged on the connection profile (38) of the manipulator tool (3) in the extended position of the rod (37), the connection between the connection member (10) and the connection profile (38) being locked by inserting the connection profile (38) into the tube (31), the connection profile (38) advantageously being elastically deformable so as to guarantee the connection member (10) is held on the connection profile (38), even before it is inserted into the tube (31).
